(19) United States
(12) Patent Application Publication
(10) Pub. No.: US 2009/0018414 A1
(43) Pub. Date: Jan. 15, 2009

(54) Subcutanous Blood Vessels Imaging System

(75) Inventor: Mehrdad Toofan, Davis, CA (US)

Correspondence Address: Mehrdad Toofan, 1603 Santa Rosa St., Davis, CA, 95616, US

(73) Assignee: Mehrdad Toofan, Davis, CA (US)

(21) Appl. No.: 11/690,518

(22) Filed: Mar. 23, 2007

(51) Int. Cl. A61B 5/103

(52) U.S. Cl. 600/310; 600/473

(57) ABSTRACT

A real time imaging system is described which displays subcutaneous veins whereby facilitating diagnosis, inspection and easy intravenous access for administration of drugs. The imaging system comprises an infrared source (1), a pinhole focusing unit (7), an infrared Focal Plane Array (8), and a display unit (10). The infrared source (1) emits infrared light which penetrates and is subsequently scattered differently at different layers and depths of the anatomical structure (11) while substantially being absorbed by the blood vessels. Reflected light (4) from the anatomical structure (11) contains the imaging information of the subcutaneous blood vessels of the anatomical structure (11) which is then focused by said pinhole focusing unit (7) upon said infrared Focal Plane Array (8). The image captured by said infrared Focal Plane Array is then displayed on the display unit (10).

### TECHNICAL FIELD

The invention described here relates in general to medical imaging systems and in particular to a system and method to facilitate the viewing of subcutaneous veins. Such visualization would permit a better diagnosis, inspection and easy intravenous access for administration of drugs.

### BACKGROUND OF THE INVENTION

Having an enhanced visualization of subcutaneous blood vessels is highly desired by those practicing in the medical field. Although subcutaneous blood vessels could easily be distinguished with the naked eyes for many patients, being able to see these veins for a large number of patients (especially for newborns and small children) constitutes a major problem for physicians for diagnosis purposes, or for those skilled in the medical art who need to perform intravenous administration.

Among many imaging techniques introduced to alleviate the problem in prior art, the most commonly referred technique relies on the optical properties of skin and subcutaneous blood vessels. See, e.g., Bashkatov, et al., “Optical properties of human skin, subcutaneous and mucous tissues in the wavelength range from 400 to 2000 nm,” J. Phys. D: Appl Phys. 38 , 2543-2555 (2005). The idea behind these techniques relies on the fact that different compounds in the skin absorb certain wavelength of incident light to various degrees. In certain infrared range, absorption is dominated by hemoglobin component of the blood, and therefore reflected light from, or transmitted light through skin carries imaging information regarding blood vessels shapes and possibly their contents. See, e.g., Nishidate, et al., “Topographic Imaging of Veins Using Reflectance Images at Isosbestic Wavelengths,” SICE, 2145-2148 (Aug. 4-6, 2004).

One imaging technique used to explore subcutaneous blood vessel is Optical Coherence Tomography (OCT). In this technique, which is based on Michelson interferometer principle, a low coherence light source is reflected off of the skin, and is then combined with portion of the source light reflected from a reference mirror to form an interference pattern. In order to get snapshots at different depths, OCT varies the optical path of the reference light in a smooth fashion through an optical delay line, while also controlling the wavelength of the incident light. While OCT provides unprecedented resolution, it suffers greatly from small field of view and relative slow frame rates. In addition, it is expensive to build an OCT.

Use of infrared to record subcutaneous veins has been known in the literature since 1930s. See, e.g., Wilson, E. “The Changes In Infrared Photographs Taken During The Treatment of Varicose Veins,” American Journal of Surgery, 37:470-474, 1937 wherein it is concluded that “The visibility of subcutaneous veins by infra-red photography, however, is dependent upon the thickness of the skin, the vein wall, and the size of the vein.”

U.S. Pat. No. 4,817,622 issued to Pennypacker, et al, discloses a method wherein infrared is used to view subcutaneous vascular structures. The disclosed apparatus consists of a CCD video camera which is connected to a video display through a video processing circuitry to a monitor screen, an objective lens through which the illuminated skin is investigated, a mirror to deflect the image, and an infrared bandpass filter. A great deal of patent's text is used to explain the video processing circuitry which is shown in more details in FIG. 3 and FIG. 4 of the above patent. Along with the complex circuitry needed to enhance the captured image, the video display is unconventional as it has to be partially transparent in order to allow the beam of light reflected from the mirror to reach the eye. Finally, the apparatus has a very small filed of view and requires a cumbersome movement of camera around manually to display various portions of skin (See FIG. 1B of the above patent).

U.S. Pat. No. 5,519,208 issued to Esparza, et al, discloses an imaging apparatus for gaining intravenous access. The invention consists of a lamp, a transmittance filter and either a reflectance filter or another transmittance filter to view the venous structures under the skin. The patent suggests that visible light is optimum for some venous system, and as such the transmittance filter needs to pass light in that range. It further advocates usage of an image intensifier when an infrared light is needed. Neither of these assertions could be scientifically supported as the visible light hardly can penetrate the skin, and image intensifiers, such as the preferred embodiment of CdS photosensors mentioned in the above patent, do not operate in the infrared range.

U.S. Pat. No. 5,947,906 issued to Dawson, et al, discloses an apparatus for viewing subcutaneous veins compromising of a light source which extends from infrared to ultraviolet, a video monitor, a CCD camera, optical filters to remove visible light, and supporting structure. The problem with the above arrangement is that light depth penetration into the skin is proportional to the wavelength, and obviously an ultraviolet light requirement of the above patent hardly supports the claim for contrast enhancements of pigmented skins of Afro-American patients. In addition, the CCD sensitivity response tails out in the ultraviolet range.

U.S. Pat. No. 6,032,070, and its continuation U.S. Pat. No. 6,272,374, and its further continuation U.S. Pat. No. 7,006,861 issued to Flock, et al, discloses apparatuses for viewing anatomical structures such as a blood vessel. In their most concise form, the elements of these patents compromise a light source, an image detector, and a monitor which receives and displays image information from said image detector. Their choice of image detector is limited to camera devices. Furthermore, their general choice of any light source to see the subcutaneous veins cannot be scientifically supported as one needs light of infrared nature to detect the blood vessels. The reason is due to the fact that light wavelengths below near infrared range can hardly penetrate the skin, or if it does, it will not be absorbed by hemoglobin content of blood, and hence no imaging is possible. The places where the nature of the light has been identified to be in infrared range, in the above reference, the source has either been limited to be monochromatic within the range of 700-900 nm, or the monitor and image detector are restricted to be part of a single unit, or the image detector and the source light are restricted to be part of the same unit, or additional elements have been added to the invention.

U.S. Pat. No. 6,230,046 issued to Crane, et al, discloses a system and method for enhancing visualization of veins. The system comprises of a light source, and a low level light detection means which is selected from the group of image intensifier tube, a photomultiplier, a photodiode, and an intensified charged coupled display. The system, however is ideally operate in the low light environment where ambient light preferably needs to be excluded as is indicated by the filter means 19 represented in FIG. 3 of the above patent. When using the light source in the infrared range, as the patent's text also reveals, light covering is needed to increase the SNR (Signal to Noise Ratio) in highly illuminated background lighting which are found in “a windowless but, highly lit operating room at a hospital”. In addition, the night vision goggles used in this invention are quite uncomfortable to wear during normal day to day operation for those working in a medical facility who need to administer drugs during intravenous access.

U.S. Pat. No. 6,178,340 issued to Svetliza, discloses a system for 3-D infrared imaging of the vascular network for puncturing the veins by a hypodermic needle. The invention discloses dual light sensors which detect the light from dual infrared LED light sources and then through a processor project the image on a display unit which would be perceived as a 3-D image by a user wearing a pair of eye glasses of different colors. However, synchronization of these dual images by the associated processor is quite impractical due to imperfections found in light sensors, the light sources and the asymmetrical position of the subject under study with respect to these elements. In addition, the usage of a fixed wavelength excludes a large number of people whose veins are at different skin depth.

U.S. Pat. No. 6,424,858 issued to Williams, discloses an imaging apparatus to provide real time image of vasculature of the human body. The invention is comprised of a curtained cabinet wherein the light from an infrared light source is first reached the subject under study through multiple fiber optic cables, then transmitted through the subject and finally picked up by a light receive apparatus which displays it on a monitor. Other elements have been added to modify either the intensity or the source of the light. The invention not only requires an environment completely dark and void of visible light, which is quite impractical due to its setup, it would require extremely sensitive light sensors to achieve a viable SNR.

### SUMMARY OF THE INVENTION

A system is disclosed which reveals real time imaging of subcutaneous blood vessels. The subject, whose veins needs to be visualized, is subject to a diffused and broad band infrared light source. Reflected waves from the subject, through a pinhole focusing unit, or alternatively through an infrared focusing lens unit, is focused upon and captured by an infrared Focal Plane Array and subsequently is displayed on a display unit. Digital Image and Signal Processing maybe applied to the output of the infrared Focal Plane Array and/or contrast enhancing elements maybe inserted in the optical path in order to increase SNR (Signal to Noise Ratio) and achieve a clearer presentation of the veins.

An object of this invention is to make the process of real time viewing of the subcutaneous blood vessels through simple and inexpensive optical elements.

Yet another objective of this invention is to make the system as compact as possible to aid the manufacturing process.

Another objective of this invention is to allow the system to be easily operated by a medical facility personnel or physician in a natural and user friendly setup.

These and other objects of the invention are achieved and appreciated by one skilled in the applicable art as a detailed description of representative embodiments thereof proceeds.

### BRIEF DESCRIPTIOIN OF THE DRAWINGS

The invention will be more clearly understood when detailed description of the invention is accompanied by referring to the accompanying drawings wherein:

FIG. 1 illustrates a preferred embodiment of the present invention wherein image of anatomical structures is captured by an infrared Focal Plane Array through a pinhole focusing unit such as a Non-Redundant Array (NRA) aperture, or a Uniformly Redundant Array (URA) aperture, or a Uniformly Distributed Array (UDP) aperture and subsequently being processed and displayed on a display unit.

FIG. 2 illustrates another preferred embodiment of the present invention wherein image of anatomical structures is captured by an infrared Focal Plane Array through an infrared objective lens and subsequently being displayed on a display unit.

FIG. 3 illustrates the single pinhole selection from a multiple available pinholes to act as the aperture for the present invention.

FIG. 4 illustrates the possibility of setting the pinhole to any desired radius to act as the aperture for the present invention.

FIG. 5 illustrates using a slit instead of a pinhole for the aperture of the present invention wherein the dimension of the slit could be changed to get the best SNR.

FIG. 6 illustrates an example of a Non-Redundant Array (NRA) as the aperture for the present invention.

FIG. 7 illustrates an example of a Uniformly Redundant Array (URA) as the aperture for the present invention.

FIG. 8 illustrates an example of a Uniformly Distributed Array (UDP) as the aperture for the present invention.

FIG. 9 illustrates yet another example of a Uniformly Distributed Array (UDP) as the aperture for the present invention.

### DESCRIPTION OF THE INVENTION

The present invention provides a system whereby one would be able to visualize the subcutaneous vein structures, their shape and locations. Such imaging system is quite useful not only for diagnosis and inspection purposes, but also for intravenous access for administration of drugs. Historically, certain segment of population would exhibit hard to find veins during a hypodermic needle injection. The invention would thus assist a medically trained person to safely administer the drug.

The invention comprises of a broadband infrared light source, a focusing unit, an infrared Focal Plane Array, and a display unit. Light emitted from the broadband infrared light source is reflected from the subject under study and is focused onto the infrared Focal Plane Array. The image created by the infrared Focal Plane Array is then displayed on the display unit after possibly some needed processing. The light source is comprised of a radiant source which emits wavelengths larger than visible end of spectrum (which usually is about 720 nm), or a source which emits light in visible spectrum as well as emitting infrared wavelengths, but the visible spectrum has been excluded from reaching the infrared Focal Plane Array by utilizing a highpass InfraRed (IR) optical filter. The infrared focusing unit is a device capable of concentrating or focusing reflected infrared light from the subject onto the infrared Focal Plane Array. The term infrared Focal Plane Array refers to a solid state device selected from but not limited to a group consisting of CMOS Focal Plane Arrays, CCD Focal Plane Arrays, InGaAs Focal Plane Arrays, InSb Focal Plane Arrays, and HgCdTe Focal Plane Arrays, and associated timing, control and readout electronics. These devices comprises of a large number of pixel elements which are sensitive to infrared radiation. The image captured by these pixel elements can then be readout to the accompanying display unit. The term “subject”, unless either explicitly defined, or obvious from the context means the biological entity (human or otherwise), whose subcutaneous veins are needed to be explored, investigated, examined, or studied.

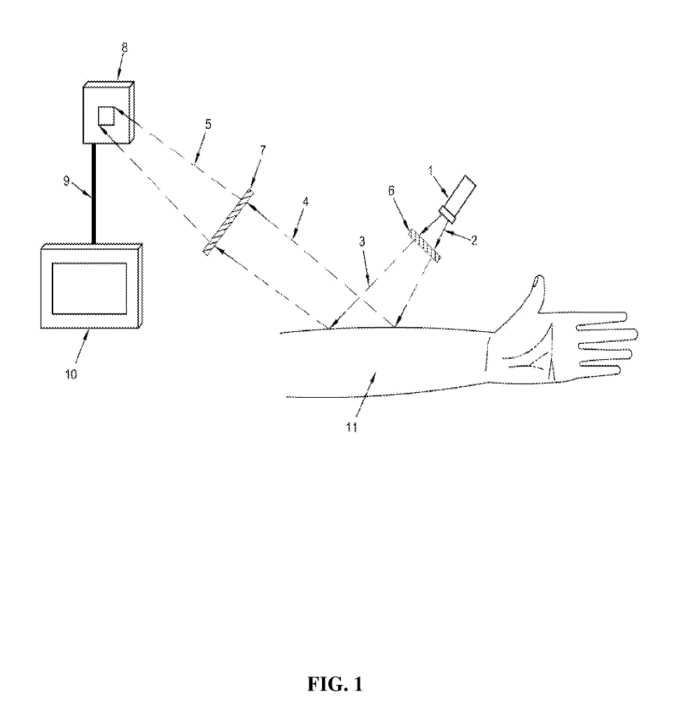

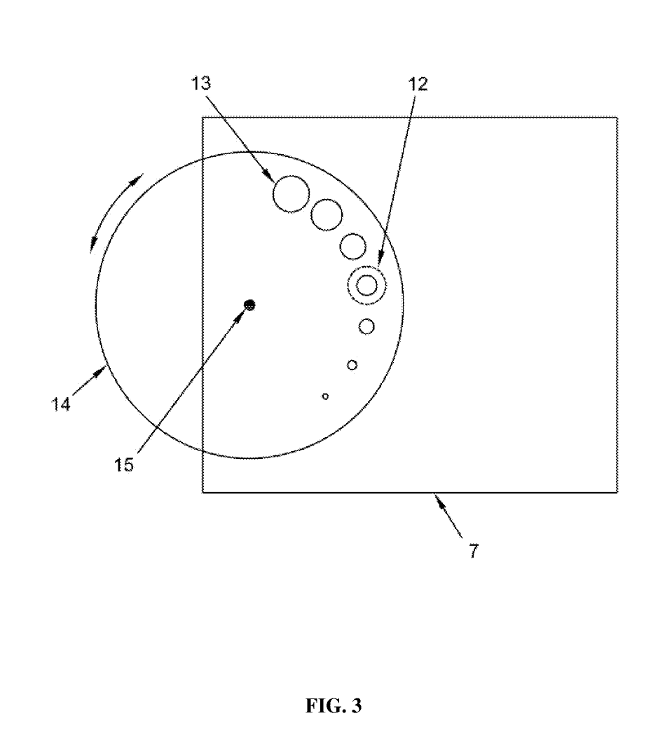

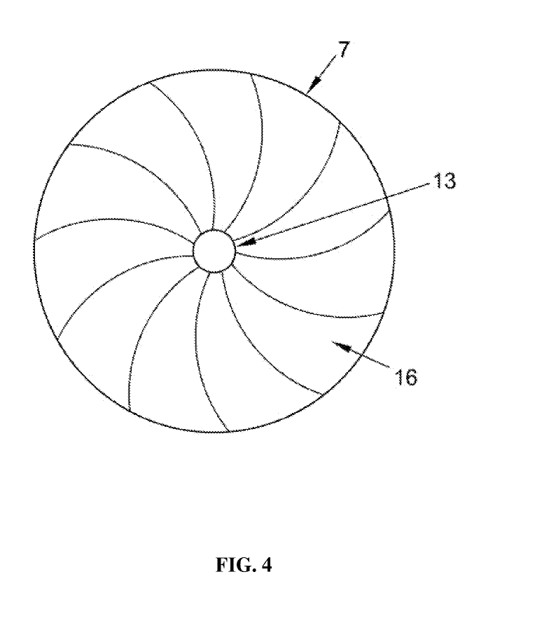

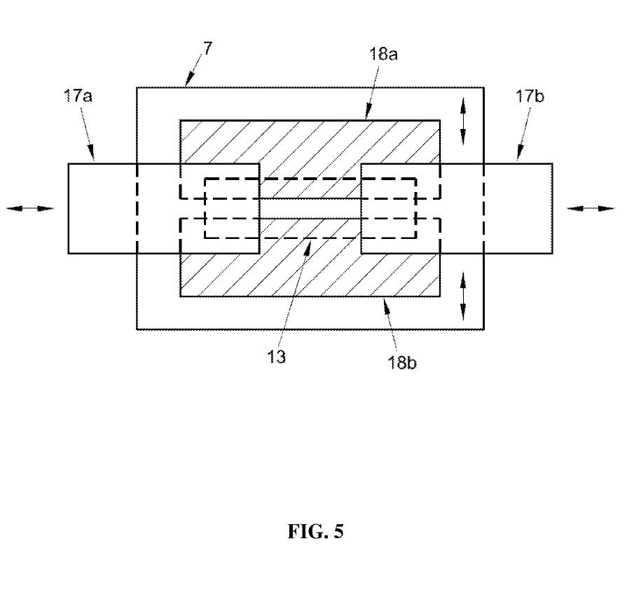

Referring to FIG. 1, a preferred embodiment of the present invention, a broad band infrared source of excitation 1 shines an infrared light on the subject under study 11. To improve the SNR of the system, the emitting infrared light 2 is further diffused by a diffuser plate 6 in form of either reflection or transmission. The emerging light 3 from the diffuser plate 6 penetrates the surface of the subject 11 and then is reflected back 4 to reach the focusing unit 7. The focusing unit 7 is an aperture comprising of either a single pinhole (as shown and discussed below for FIGS. 3 and 4), a slit (as shown and discussed below for FIG. 5), or a multiple of pinholes in form of Non-Redundant Array (NRA), Uniformly Redundant Array (URA), or Uniformly Distributed Array (UDP). The infrared light 5 which passes through the focusing unit 7 then reaches the infrared Focal Plane Array 8. Image of anatomical structures, such as veins are then transmitted via link 9 to the display unit 10. If the focusing unit 7 is compromised of either NRA or URA, or UDP, the captured imaged by the infrared Focal Plane Array 8 needs to be processed or decoded by the display unit before presenting the final image to the user of the invention. The display unit may also perform some Digital Signal Processing (DSP) operations on the captured image to improve the SNR.

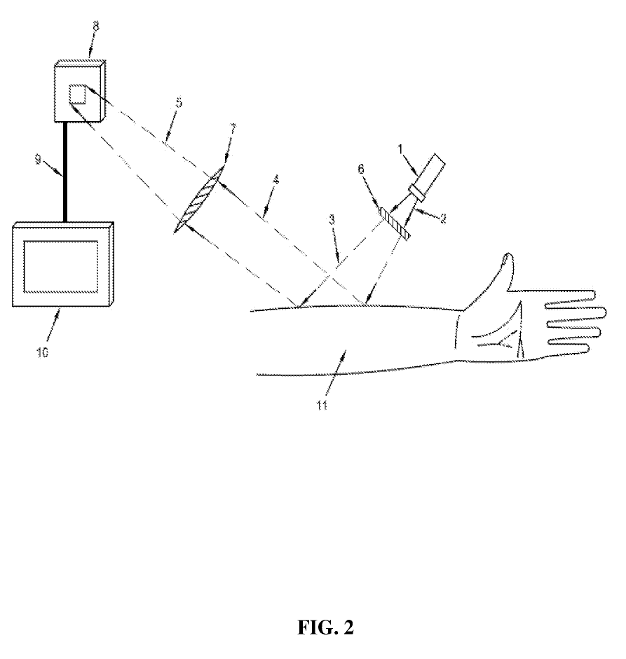

FIG. 2 illustrates another embodiment of the present invention. FIG. 2 shows a broad band infrared source of excitation 1 which shines an infrared light on the subject under study 11. To improve the SNR of the system, the emitting infrared light 2 is further diffused by a diffuser plate 6 in form of either reflection or transmission. The emerging light 3 from the diffuser plate 6 penetrates the surface of the subject 11 and then is reflected back 4 to reach the infrared focusing unit 7. The infrared focusing unit 7 comprises of an infrared objective lens which focuses the infrared light 4 on to the infrared Focal Plane Array 8. The infrared light 5 which is output of the infrared focusing unit 7 subsequently reaches the infrared Focal Plane Array 8. Image of anatomical structures, such as veins are then transmitted via link 9 to the display unit 10. The display unit may subsequently perform some DSP operations on the captured image to improve the SNR before presenting the final image to the user of the invention.

Choice of radiation source for this invention may need to obey a few noticeable requirements among many. The light source (element 1 of FIGS. 1 and 2) emits a broadband infrared radiation. In fact, all black bodies radiate infrared at certain temperatures. Among examples for broadband infrared radiation one could mention a Tungsten Halogen bulb such as Philips BC60CP19/HAL/CL 120V, or a photographic lamp, or an incandescent lamp. These bulbs have broadband spectrum, with adequate intensity, from about 300 nm well through infrared range of interests. They also satisfy a second requirements for being inexpensive, easy to operate, and maintain. They do not further require special FCC licensing for operation in a system. Broadband light could also be generated by using superluminescent diodes or lasers with very short pulses with highpass IR filter applied to the Focal Plane Array to eliminate the visible light.

LED (Light Emitting Diode) could also be used as an infrared source of illumination but care should be practiced to have the spectrum of the LED as wide as possible and the LED be of sufficient intensity. The reasons are that the spectrum of these devices normally is very narrow, and hence the light penetration into the body is limited to the chosen central wavelength of the LED. The second drawback is that their intensity is limited. One may use an array of LEDs to overcome this particular shortcoming, but then physical arrangement of these LED must be carefully considered as the collective light pattern of these identical elements adhere to the interference laws of physics and shine a patterns of dark and light bands at a distant.

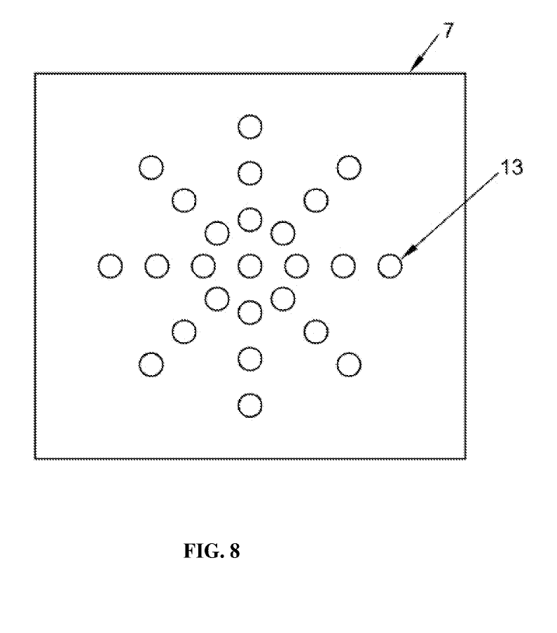

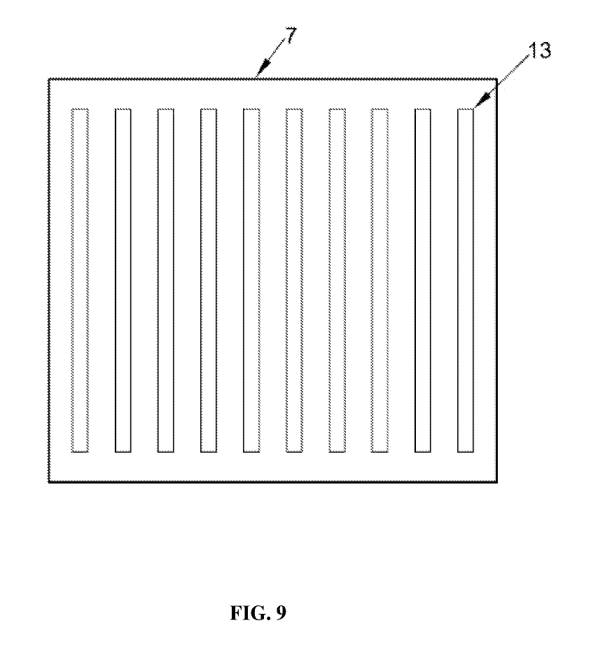

Diffused light could be used to improve the SNR. High intensity light which reaches the pixels of the infrared Focal Plane Arrays will saturate them to the point where the pixel values no longer carry valuable image information. A diffused light could be easily generated by illuminating the subject from an angle, or transmitting the light through a diffusion sheet (element 6 of FIGS. 8 and 9), or simply reflecting it off a diffusion surface. It is also possible to provide a manual dim switch in addition of, or as a replacement for the light diffuser. This arrangement will allow the operator of the system to set optimum light intensity for different subjects to get the best SNR. The diffusion sheet or surface may also be eliminated if polarizers are used to enhance the SNR of the system, or as mentioned above if the subject is illuminated from an angle, or by using a dim switch.

Infrared Focal Plane Array (element 8 of FIGS. 8 and 9) used in this invention could be based on several possibilities such as CMOS, CCD, InGaAs, InSb, and HgCdTe. CMOS or CCD Focal Plane Arrays normally exhibit some sensitivity to the infrared light, while InGaAs, InSb, and HgCdTe Focal Plane Arrays normally operate in the infrared region. For this reason, typical CCD and CMOS Focal Plane Arrays based video cameras normally use InfraRed (IR) cutoff filter to limit the camera response to the visible light. For these cameras, should they provide night shot visioning, a number of other limitations and restrictions (such as aperture or timing) are applied to the camera. Focal Plane Arrays used in this invention thus need to emphasize the infrared sensitivity, which is measured in terms of its quantum efficiency, when exposed to infrared illumination. Some techniques currently used to enhance the infrared sensitivity of these Focal Plane Arrays are reviewed below.

Front illuminated CCD Focal Plane Arrays has a maximum quantum efficiency of about 40% at 700 nm, rolling down quickly to zero at about 1100 nm. Since spectral response of a photodiode onboard a Focal Plane Array is correlated to its silicon-impurity doping and depletion depth, to improve infrared response of CCD Focal Plane Arrays, they are made of relatively thick substrate (in the order of 40-50 um) on high resistivity silicon containing much lower impurity than normal. Further improvement is achieved by using anti-reflection coating for back illumination devices to avoid fringing phenomena. As this method (usually referred to as “deep depletion” in the industry) may turned out to be costly, alternative methods such as “fully depletion” technology has become more popular wherein very thick silicon substrate (in the order of 300 um) is used to achieve a quantum efficiency near 85% at 1000 nm (See. e.g. http://design.lbl.gov/ccd).

CMOS Focal Plane Arrays, in general, have higher infrared quantum efficiency compared to their CCD counterparts. This is generally due to vertical antiblooming overflow structures which are used in CCDs to prevent migration of electrons from an oversaturated pixel to the neighboring pixels. The CMOS Focal Plane Array devices typically show quantum efficiencies of about 40% for an extended range of infrared (700 nm-850 nm), rolling down gradually to 15% at 950 nm, and further down to about zero at 1100 nm. The quantum efficiency of these devices in the infrared region could be increased, for example, by increasing the depth of N+ and P+ layers and/or usage of N well/P substrate photodiodes (See, e.g., Ardeshirpour, et al, “Evaluation of complementary metal-oxide semiconductor based photodetectors for low-level light detection,” J. Vac. Sci. Technol. A, Vol. 24, No. 3, May/Jun 2006). Furthermore, Bayer filters are typically fabricated on the top of photodiode components of these solid state Focal Plane Arrays to introduce color. These filters are composed of chess board patterns of RGB color filters, with the dominant green color. Consequently, the effective sensitivity of the infrared Focal Plane Arrays to infrared is greatly reduced. It is thus possible to increase the infrared sensitivity of these devices by either eliminating altogether the Bayer filters, or by introducing a high pass IR filter, with cutoff frequency at about 700 nm, in place of these filters.

The invention could further benefit from newly offered Focal Plane Arrays where a hybrid of CMOS and CCD has been devised to take advantage of both Focal Plane Arrays technologies. It is a well known fact that CMOS Focal Plane Arrays have some advantages wherein CCD cannot compete. For example, there is flexibility on the readout of CMOS devices due to their random access pixels; CMOS Focal Plane Arrays are much less power hungry and very high speed combined with low noise readout is possible for these type of devices; there is no need for shutter; continuous light observation is possible; and most importantly integration of controller logic, and various digital image and signal processing schemes on the same die as the CMOS Focal Plane Array could make the notion of “camera-on-a-chip” a reality. Despite these advantages, CCDs offers some great advantages such as 100% fill factors, high dynamic range, and a proven mature technology. For this reason, some manufacturers have attempted at manufacturing a hybrid CCD/CMOS combination wherein high imaging qualities of the CCD are combined with the high speed, low power, and low noise capabilities of a dedicated CMOS readout(See, e.g. Liu, et al, “CCD/CMOS Hybrid FPA for Low Light Level Imaging,” Published by Fairchild Imaging).

InGaAs Focal Plane Arrays, however, in contrast to typical CMOS and CCD Focal Plane Arrays dominantly operate in the infrared region. By altering the composition of alloys used in InGaAs absorption layer, manufacturing of room temperature operated cameras at various infrared cutoff wavelength is possible (See, e.g., Sensors Inc. SU320uSVIS-1.7RT InGaAs camera which operates from 400 nm up to 1.7 um).

The present invention, furthermore, assumes presence of an infrared focusing unit (element 7 of FIGS. 1 and 2) in order to focus the reflected infrared light from the subject under study onto the infrared Focal Plane Array. The infrared focusing unit could comprise of a pinhole through which the reflected light is focused onto the infrared Focal Plane Array. Compared to lenses, pinhole optics has the advantage of a large depth of field, lacks distortion normally seen when using lenses, as well as the fact that, if not careful, lenses could absorb light in the infrared range. Pinhole optics, however, suffers from an un-sharp image, and requires a high f/stop. Multiple pinholes of different dimensions could be used to achieve the best image qualities under different lighting conditions and when dealing with different subjects. Aperture 7 shown in FIG. 3 illustrates this notion. As shown in FIG. 3, the user of the invention could select a single pinhole 13 of the desired size (among multiple, but fixed number, of pinholes of different sizes) by rotating the aperture selection plate 14 against the pivot point 15 to align the pinhole 13 and the hole 12 to achieve the best image quality. Alternatively, the aperture size 13 could be of variable dimension, such as the one shown in FIG. 4, whose size could be either mechanically and/or electronically controlled through changing the shape of segments 16; a very common practice in almost all cameras available on the market. Alternatively, as shown in FIG. 5, a slit 13 instead of a single pinhole could be used as the aperture to project the subject's image on the infrared Focal Plane Array. In this alternative, the dimension of the slit 13, as shown in FIG. 5, could be changed by the user by opaque windows 18a, 18b, and 17a, 17b to achieve the best SNR.

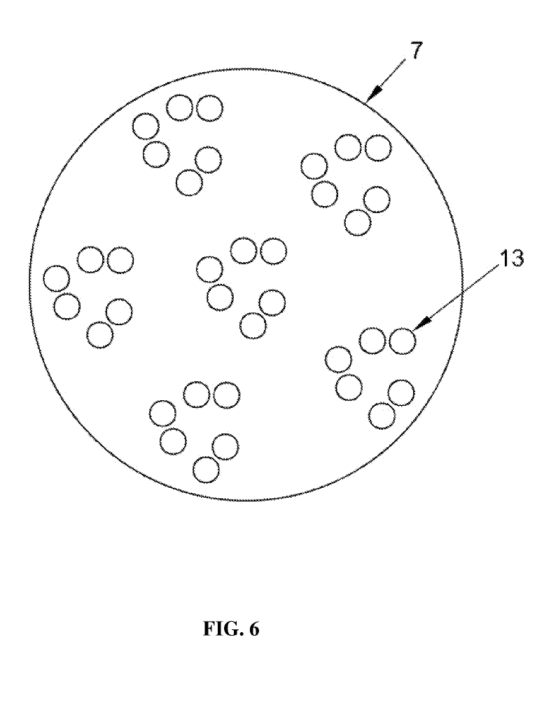

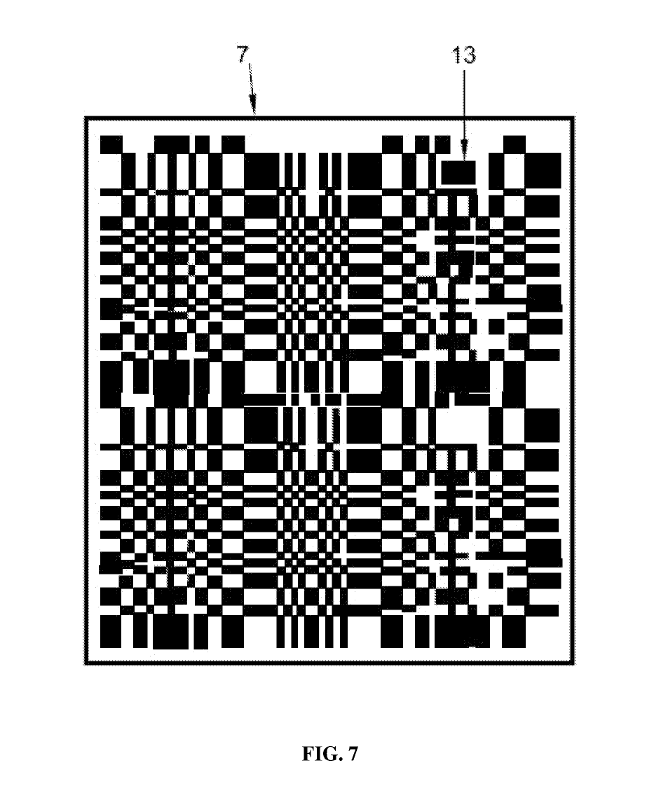

In a preferred embodiment of invention, the suggested pinhole of the infrared focusing unit could be replaced by an array of pinholes placed on an aperture in a form of either a Non-Redundant Array (NRA) (See, e.g. Meng, et al, “A Gamma Ray Imager Using Clustered Non-Redundant Array Coded Aperture”, Nuclear Science Symposium Conference Record, Vol. 2, pp 763-766, October 2003), or Uniformly Redundant Array (URA) (See, e.g., Fenimore, “Coded aperture imaging with uniformly redundant arrays”, Appl. Optics, Vol. 17, No. 3, Feb. 1, 1978, pp 337-347), or Uniformly Distributed Pinholes (UDP). UDP operates on a similar idea as in Sparse Aperture Imaging (See, e.g., Miller, et al, “Resolution Enhanced Sparse Aperture Imaging”, IEEEAC paper #1406, Version 3, pp 1-16, October 2005) relying on the fact that effective pupil diameter, which is directly proportional to the resolution of a diffraction limited image, is increased by using uniformly distributed pinholes or slits across the aperture plate. In a Non-Redundant Array, an example of which is shown in FIG. 6, no two array elements (pinholes) have the same spacing. In a URA, an example of which is shown in FIG. 7, separation between pairs of holes occur a constant number of times. The Uniformly Distributed Pinholes, examples of which are shown in FIGS. 6 and 7, is comprised of multiple pinholes or slits positioned at fixed distances from each other so that the whole ensemble forms a particular pattern.

One advantage of this embodiment (wherein either one of NRA, URA, or UDP has been selected for the aperture) compared to a single pinhole approach, is that if there are N pinholes in the aperture, the SNR of the system would theoretically increase by a factor of √N (square root of N). Another advantage of this embodiment is that the incident broadband infrared light is reflected from different anatomical layers located at different depths. When seen from the aperture, they represent light coming from objects at different depth of fields. These objects, which are at different distances from the aperture, will cast shadows of different overall sizes of the aperture onto the infrared Focal Plane Array. Hence, slices of the image at different depth can be reconstructed by “treating the picture as it was formed by an aperture scaled to the size of the shadow produced by the depth under consideration.” A three dimensional (3D) image of the anatomical structure under study is thus possible by constructing images at different depths. Alternatively, high resolution image of an anatomical structure at a particular depth could be displayed. It is noted that the captured image on the infrared Focal Plane Array, however, represents an overlap of multiple images of the object under study. Therefore, reconstruction of the image of the original object requires subsequent processing of the image captured by the infrared Focal Plane Array. Furthermore, if the infrared Focal Plane Array has not been equipped with a high-pass IR filter to filter out the visible light, such a filter would be necessary to be placed either before or after the aperture.

Yet in another embodiment of the present invention, the infrared focusing unit is comprised of an objective lens. Reflected infrared light from the subject is focused by the objective lens onto the infrared Focal Plane Array. In order to improve SNR of the imaging system, however, attempt must be made to make the absorption of infrared light by the infrared focusing unit to a minimum while at the same time filtering out the visible light. The objective lens, which is noted in this embodiment as an infrared lens, could be made from, but not limited to, materials such as ZnS, ZnSe, GaAs, Ge, poly ethylene (PE) plastics, and chalcogenide glass. If the material used in construction of the infrared lens does not exhibit the tendency to eliminate the visible light, the surface of the lens could be coated with an IR filter material to block out the visible light. Alternatively, one may use a thin Fresnel fraction lens as an infrared lens. The “POLY IR 6” Fresnel lens manufactured by Fresnel Technologies, Inc. is an example that could be used for this purpose. Anther example of a thin infrared Fresnel lens is the disclosure of an invention by Lee, et al, in the U.S. Pat. No. 6,493,155.

The present invention, furthermore, assumes presence of a display unit (element 10 of FIGS. 1 and 2). The image captured by the infrared Focal Plane Array will be displayed on this unit. The display unit, as is discussed below, has some associated software/hardware to transfer (via link 9 shown in FIGS. 1 and 2) from the infrared Focal Plane Array the image that is captured there and then possibly to process the image. The display unit, further has means to display the image on an LCD, a digital image projector, a plasma screen, or alike for viewing. The display unit could thus be physically attached through high speed links (such as Gigabit Ethernet, USB, Firewire, etc.) to the infrared Focal Plane Array. Alternatively, the display unit could be physically a separate entity from the infrared Focal Plane Array and use wireless technology to link to the infrared Focal Plane Array. Therefore, the display unit could be either integrated with the infrared focusing unit and the infrared Focal Plane Array to form a compact design, or alternatively, the display unit could be a separate entity (such as a Personal Computer) from the other two and link to them via wire, or via wireless technology.

For the embodiment of the present invention wherein a pinhole, a slit, or an infrared objective lens is used as the focusing element, the display unit may require some Digital Image and Signal Processing of the captured image to improve the SNR.

If the embodiment, however, uses Non-Redundant Array (NRA), Uniformly Redundant Array (URA), or Uniformly Distributed Array (UDP), as explained before for the focal unit, then the image would need to be processed or decoded further before being displayed. In the case of URA, for example, the captured image by the infrared Focal Plane Array satisfies the correlation equation (See, e.g., Accorsi, et al, “A Coded Aperture for High-Resolution Nuclear Medicine Planar Imaging with a Conventional Anger Camera: Experimental Results”, Nuclear Science Symposium Conference Record, 2001 IEEE, vol. 3, pp 1587-1591):

R=OXA (1)

where R is the captured image by the infrared Focal Plane Array, O is the object function, A is the aperture function and X represents the correlation operator. By choosing an appropriate aperture (A), a decoding pattern G could be constructed such that the periodic correlation A*G is a delta function (δ). That is, a perfect image of O can be constructed by applying a periodic correlation (*) of R with G:

R*G=(OXA)*G=OX(A*G)=OX δ=O (2)

The required processing power needed for the NRA, URA, or UDP implementation of the present invention, presents the possibility of implanting various Digital and Signal Processing (DSP) algorithms to further enhance the SNR of the reconstructed image. In addition, as mentioned before, the same processing power could be used to one's advantage to present the 3D image of the anatomical structure under study to the user.

The processing power and/or DSP needed for the construction of the image in case of the NRA, URA, or UDP implementation of the present invention or for other embodiments presented by this disclosure could be implemented all in software on a Personal Computer (PC), or a dedicated general purpose/DSP Processor. In fact, if a PC is used to process the captured images from the infrared Focal Plane Array, it could as well be used to display and record the captured images. Alternatively, this processing power and/or DSP could be implemented through the usage of dedicated hardware chips such ASICs (Application Specific Integrated Circuits), FPGA (Field Programmable Gate Array), and/or firmware. Alternatively, the user may use a combination of the above two methods (software and hardware) to achieve the desired results.

The SNR of the image captured by the infrared Focal Plane Array maybe further improved by using a pair of InfraRed (IR) polarizers in the present invention. A good portion of incident infrared light is reflected from the skin interface of the subject and thus carries no desired imaging information. To eliminate the infrared light reflected immediately from the skin, one infrared polarizer will be situated in front of the infrared light source (after element 6 of FIGS. 1 and 2). The second infrared polarizer maybe placed either before, or after the infrared focusing unit (element 7 of FIGS. 1 and 2). While the optical axis of polarization of the first polarizer is fixed, the second polarizer could be manually rotated, thus changing the polarization axis of the second polarizer with respect to the first one, to give the operator of the present invention the best image quality on the display unit.

The SNR of the image captured by the infrared Focal Plane Array maybe further improved by exposing the subject under study to direct heat( such as an electric heater), or indirect heat (such as hot air), or by applying pressure to the subject under study using a Tourniquet for example.

The invention has been described in details with particular reference to certain preferred embodiments thereof, but it will be understood that variations and modifications can be effected within the sprit and scope of the invention.

What is claimed:

1. A real time imaging system displaying subcutaneous features comprising an infrared source illuminating an area of interest; a pinhole focusing unit; an infrared Focal Plane Array and a display unit, whereby:
said pinhole focusing unit consists of one or more pinholes on an aperture wherein said pinhole focusing unit focuses light from the area of interest upon said infrared Focal Plane Array; and
said display unit processes and displays captured data from said infrared Focal Plane Array.

2. The system of claim 1 wherein said infrared source is selected from, but not limited to, a halogen lamp, a photographic lamp, an incandescent lamp, or an LED, or a superluminescent diode, or a laser with very short pulses.

3. The system of claim 1 wherein said pinhole focusing unit is a Non-Redundant Array, or a Uniformly Redundant Array, or a Uniformly Distributed Array.

4. The system of claim 1 wherein said focusing unit is a pinhole or a slit of sufficient size.

5. The system of claim 1 wherein said infrared Focal Plane Array is selected from a group comprising of CMOS, CCD, InGaAs, InSb, and HgCdTe Focal Plane Arrays.

6. The system of claim 1, further comprising an IR polarizer.

7. The system of claim 1, further comprising an FPGA, or an ASIC, or a general-purpose processor, or a general-purpose digital signal processor.

8. The system of claim 1 wherein said display unit is a personal computer.

9. The system of claim 1 wherein wireless technology is used to transfer data, or control signals between said infrared Focal Plane Array and said display unit.

10. The system of claim 1 wherein heat or pressure, or a combination thereof, is used to enhance the SNR.

11. The system of claim 1 wherein the intensity of said infrared source can be varied.

12. A real time imaging system displaying subcutaneous features comprising an infrared source; an infrared lens focusing unit; an infrared Focal Plane Array and a display unit, whereby:
said infrared source is a broadband, or diffused infrared, or a combination thereof source of excitation which would illuminate an area of interest; and
said infrared lens focusing unit is an infrared lens wherein said infrared lens focusing unit focuses light from the area of interest upon said infrared Focal Plane Array; and
said display unit processes and displays captured data from said infrared Focal Plane Array.

13. The system of claim 12 wherein said infrared source is selected from, but not limited to, a halogen lamp, a photographic lamp, an incandescent lamp, or an LED, or a superluminescent diode, or a laser with very short pulses.

14. The system of claim 12 wherein said infrared lens focusing unit is made from, but not limited to, ZnS, ZnSe, GaAs, Ge, poly ethylene plastics, and chalcogenide glass.

15. The system of claim 12 wherein said infrared Focal Plane Array is selected from a group comprising of CMOS, CCD, InGaAs, InSb, and HgCdTe Focal Plane Arrays.

16. The system of claim 12, further comprising an IR polarizer.

17. The system of claim 12, further comprising an FPGA, or an ASIC, or a general-purpose processor, or general-purpose digital signal processor.

18. The system of claim 12 wherein said display unit is a personal computer.

19. The system of claim 12 wherein wireless technology is used to transfer data, or control signals between said infrared Focal Plane Array and said display unit.

20. The system of claim 12 wherein heat or pressure, or a combination thereof, is used to enhance the SNR.

21. The system of claim 12 wherein the intensity of said infrared source can be varied.

22. A method to display the real time image of subcutaneous features comprising the steps of:
(a) providing an infrared source illuminating an area of interest;
(b) focusing light from the area of interest upon an infrared Focal Plane Array by a pinhole focusing unit; and
(c) generating an image from data measured by said infrared Focal Plane Array and displaying the image.

23. The method of claim 22 wherein said infrared source is selected from, but not limited to, a halogen lamp, a photographic lamp, an incandescent lamp, or an LED, or a superluminescent diode, or a laser with very short pulses.

24. The method of claim 22 wherein said pinhole focusing unit is a Non-Redundant Array, or a Uniformly Redundant Array, or a Uniformly Distributed Array.

25. The method of claim 22 wherein said focusing unit is a pinhole or a slit of sufficient size.

26. The method of claim 22 wherein said infrared Focal Plane Array is selected from a group comprising of CMOS, CCD, InGaAs, InSb, and HgCdTe Focal Plane Arrays.

27. The method of claim 22 wherein said display unit is a personal computer.

28. A method to display the real time image of subcutaneous features comprising the steps of:
(a) providing an infrared source illuminating an area of interest;
(b) focusing light from the area of interest upon an infrared Focal Plane Array by an infrared lens focusing unit; and
(c) generating an image from data measured by said infrared Focal Plane Array and displaying the image.

29. The method of claim 27 wherein said infrared source is selected from, but not limited to, a halogen lamp, a photographic lamp, an incandescent lamp, or an LED, or a superluminescent diode, or a laser with very short pulses.

30. The method of claim 27 wherein said infrared lens focusing unit is made from, but not limited to, ZnS, ZnSe, GaAs, Ge, poly ethylene plastics, and chalcogenide glass.

31. The method of claim 27 wherein said infrared Focal Plane Array is selected from a group comprising of CMOS, CCD, InGaAs, InSb, and HgCdTe Focal Plane Arrays.

32. The method of claim 27 wherein said display unit is a personal computer.
